(19) United States
(12) Patent Application Publication
(10) Pub. No.: US 2023/0200732 A1
(43) Pub. Date: Jun. 29, 2023

(54) FIXING STRUCTURE OF TWO WATCHBANDS

(71) Applicants: Fasikl Incorporated, Dallas, TX (US); Hangzhou Fasikl Technology Co.,Ltd, Hangzhou (CN)

(72) Inventors: Bing Ye, Hangzhou (CN); Baitong Wang, Hangzhou (CN)

(73) Assignees: Fasikl Incorporated, Dallas, TX (US); Hangzhou Fasikl Technology Co.,Ltd, Hangzhou, ZJ (CN)

(21) Appl. No.: 18/118,135

(22) Filed: Mar. 7, 2023

(51) Int. Cl. A61B 5/00

(52) U.S. Cl. CPC A61B 5/681; A61B 5/6831

(57) ABSTRACT

The present disclosure discloses a fixing structure of two watchbands, including a main unit, an inner band and an outer band, wherein the inner band is connected with a bottom of the main unit; the outer band is rotatably connected with one side of the main unit; a groove is arranged on an outer side of the inner band; a hook surface is arranged in the groove; a loop surface matched with the hook surface is arranged on an inner side of the outer band; and the hook surface is adhered to the loop surface. In the present disclosure, by the arrangement of the hook surface and the loop surface, the hook surface and the loop surface are adhered, so that the inner band and the outer band can overlap each other to be fixed, which avoids the inner band from shifting to affect the use effect.

### TECHNICAL FIELD

The present disclosure relates to the technical field of intelligent medical wearable devices, specifically to a fixing structure of two watchbands.

### BACKGROUND

With the development of science and technology and the improvement of the medical level, medical intelligent wearable devices are more and more popular. For example, a wristband nerve stimulator that can regulate the percutaneous nerves to treat neuralgia and primary tremor can be worn on the wrist like a watch, and the corresponding nerves distributed in the wrist can be stimulated by a current generated by electrodes, so as to achieve the purpose of treatment. For example, a wristband blood pressure recorder that can measure a blood pressure is an electronic blood pressure meter that can be worn on the wrist for a long time like a watch. It mainly measures a blood pressure through a blood pressure sensor and continuously measures the blood pressure.

The wristband nerve stimulator includes two watchbands overlapping inside and outside. The inner watchband is a watchband embedded with a plurality of electrodes, and the outer watchband is a band used for fixing the stimulator instrument on the wrist.

The wristband blood pressure recorder includes two watchbands overlapping inside and outside. The inner watchband is an airbag watchband, and the outer watchband is a band used for fixing the recorder instrument on the wrist.

The medical wearable device of a two-watchband structure requires that overlapping positions of the inner watchband and the outer watchband is relatively unchanged during use. The inner watchband is a consumable and has a short service life, which requires that the inner watchband and the outer watchband can be easily mounted and removed.

In the existing two-watchband structure, the inner watchband and the outer watchband are not relatively fixed. Since both the inner watchband and the outer watchband are flexible bodies, improper wearing during the use is likely to cause the relative position of the inner watchband and the outer watchband to shift, which will seriously affect the treatment or measurement effect. Or in the existing two-watchband structure, the inner watchband and the outer watchband are fixed by clamping a buckle into a hole location. This method is more complex, cannot be infinitely adjusted, and has poor wearing comfort.

### SUMMARY

The present disclosure aims to provide a fixing structure of two watchbands, which solves the problems in the background section.

In order to achieve the above objective, the present disclosure provides the following technical solution: a fixing structure of two watchbands, including a main unit, an inner band and an outer band, wherein the inner band is connected with a bottom of the main unit; the outer band is rotatably connected with one side of the main unit; a groove is arranged on an outer side of the inner band; a hook surface is arranged in the groove; a loop surface matched with the hook surface is arranged on an inner side of the outer band; and the hook surface is adhered to the loop surface.

Preferably, a hanger is arranged on one side of the main unit opposite to the outer band.

Preferably, the inner band is one of an electrode band, an airbag watchband, a silica gel watchband or a rubber watchband.

Preferably, the groove has a thickness of 0.1 mm to 1 mm.

Preferably, a material of the outer band is one of a nylon fabric, rubber, silica gel or leather.

Compared with the prior art, the present disclosure has the beneficial effects as follows:

In the present disclosure, by the arrangement of the hook surface and the loop surface, the hook surface and the loop surface are adhered, so that the inner band and the outer band can overlap each other to be fixed, which avoids the inner band from shifting to affect the use effect. The outer band can fix various types of inner bands, so that quick mounting and removal of the outer band and the inner band can be achieved, and the device is more convenient and more stable in a use process.

### BRIEF DESCRIPTION OF THE DRAWINGS

FIG. 1 is a schematic structural diagram I of the present disclosure;

FIG. 2 is an exploded schematic structural diagram of an inner band of the present disclosure;

FIG. 3 is a schematic structural diagram of an outer band of the present disclosure; and

FIG. 4 is a schematic structural diagram II of the present disclosure.

### DETAILED DESCRIPTION OF THE EMBODIMENTS

The technical solutions in the embodiments of the present disclosure will be described clearly and completely below with reference to the drawings in the embodiments of the present disclosure. Obviously, the embodiments described herein are only part of the embodiments of the present disclosure, not all the embodiments. Based on the embodiments in the present disclosure, all other embodiments obtained by those of ordinary skill in the art without creative work shall fall within the protection scope of the present disclosure.

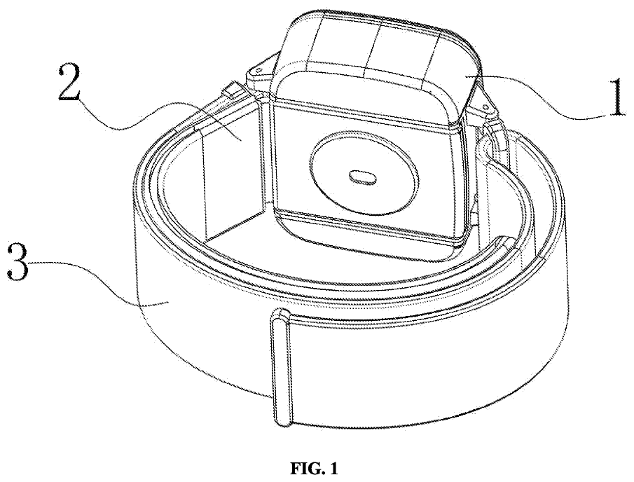

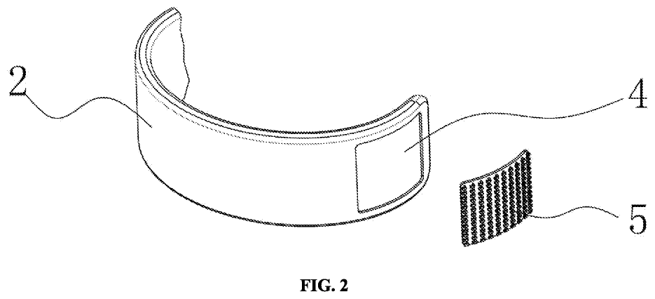

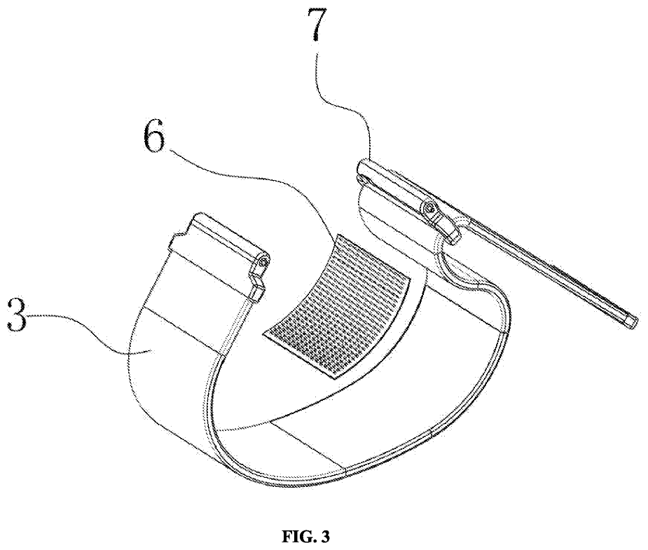

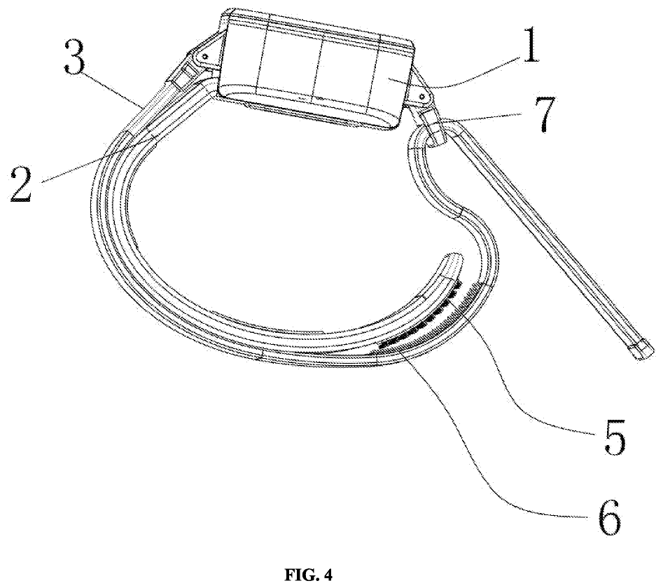

Referring to FIG. 1 to FIG. 4, one embodiment provided by the present disclosure is shown: A fixing structure of two watchbands includes a main unit 1, an inner band 2 and an outer band 3. The inner band 2 is connected with a bottom of the main unit 1. The outer band 3 is rotatably connected with one side of the main unit 1. A groove 4 is arranged on an outer surface of a tail end of the inner band 2. The groove 4 has a thickness of 0.1 mm to 1 mm. A hook surface 5 is arranged in the groove 4. A loop surface 6 matched with the hook surface 5 is arranged on an inner surface of the outer band 3. The hook surface 5 is adhered to the loop surface 6, so that the inner band 2 and the outer band 3 can overlap each other to be fixed, and the outer band 3 can fix various types of inner bands 2, which can achieve quick mounting and removal of the outer band 3 and the inner band 2. The device is more convenient and more stable in a use process. By the adoption of the fixing manner, the inner band 2 and the outer band 3 can be firmly stacked, which avoids the inner band 2 from shifting to affect the use effect.

Specifically, a hanger 7 is arranged on one side of the main unit 1 opposite to the outer band 3. The outer band can pass through the hanger 7. A distance between the outer band 3 and the hanger 7 is adjusted to adapt to a user.

Specifically, a number of small elastic hooks are aranged on the hook surface 5, and many small loops formed by fluff are arranged on the loop surface 6. When the inner band 2 and the outer band 3 overlap, overlapping portions of the hook surface 5 and the loop surface 6 are slightly pressed to fix the overlapping tail ends of the inner band 2 and the outer band 3.

Specifically, the inner band 2 of this embodiment is an airbag watchband. The inner band 2 is not limited to the airbag watchband. The inner band 2 can also be an electrode watchband and a flexible material such as silica gel or rubber.

Specifically, the hook surface 5 is an ultrathin plastic material. The hook surface 5 is adhered in the groove 4 of the inner band 2 through glue. After adhesion, an outer surface of the hook surface 5 is slightly higher than a surface of the inner band 2.

Specifically, when a material of the outer band 3 is a nylon fabric, a corresponding region of an inner surface of the outer band 3 can be made into the loop surface 6 by a process. The loop surface 6 can be fitted and fixed with the outer band 3 at the corresponding region of the inner surface of the outer band 3 by means of stitching by a machine, or the loop surface 6 can also be adhered to a corresponding position of the outer band through glue.

Further, when the material of the outer band 3 is rubber, silica gel or leather, the loop surface 6 can be fitted and fixed with the outer band at the corresponding region of the inner surface of the outer band 3 by glue.

Specifically, the loop surface 6 is slightly wider than the hook surface 5, and the loop surface 6 is longer than the loop surface 5.

Specifically, the inner band 2 can be in a size of L, M or S.

For those skilled in the art, it is apparent that the present disclosure is not limited to the details of the demonstrative embodiments mentioned above, and that the present disclosure can be realized in other specific forms without departing from the spirit or basic features of the present disclosure. Therefore, from any point of view, the embodiments should be regarded as exemplary and non-limiting. The scope of the present disclosure is defined by the appended claims rather than the above description. Therefore, all changes falling within the meanings and scope of equivalent elements of the claims are intended to be included in the present disclosure. No drawing markings in claims shall be deemed to limit the claims involved.

What is claimed is:

1. A fixing structure of two watchbands, comprising a main unit, an inner band and an outer band, wherein the inner band is connected with a bottom of the main unit; the outer band is rotatably connected with one side of the main unit; a groove is arranged on an outer side of the inner band; a hook surface is arranged in the groove; a loop surface matched with the hook surface is arranged on an inner side of the outer band; and the hook surface is adhered to the loop surface.

2. The fixing structure of the two watchbands according to claim 1, wherein a hanger is arranged on one side of the main unit opposite to the outer band.

3. The fixing structure of the two watchbands according to claim 1, wherein the inner band is one of an electrode band, an airbag watchband, a silica gel watchband or a rubber watchband.

4. The fixing structure of the two watchbands according to claim 1, wherein the groove has a thickness of 0.1 mm to 1 mm.

5. The fixing structure of the two watchbands according to claim 1, wherein a material of the outer band is one of a nylon fabric, rubber, silica gel or leather.
